(19) United States
(12) Patent Application Publication
(10) Pub. No.: US 2023/0326614 A1
(43) Pub. Date: Oct. 12, 2023

(54) REMOTE MEDICAL PROCTORING SYSTEM AND METHOD THEREOF

(71) Applicant: POINT 2 POINT PRODUCTION LTD., Tzur Hadassah (IL)

(72) Inventor: Erez KURIEL, Tzur Hadassah (IL)

(21) Appl. No.: 18/209,626

(22) Filed: Jun. 14, 2023

Related U.S. Application Data
(63) Continuation-in-part of application No. PCT/IL2022/050178, filed on Feb. 15, 2022.
(60) Provisional application No. 63/125,403, filed on Dec. 15, 2020.

(51) Int. Cl. G16H 80/00; G16H 40/67; G16H 50/20

(52) U.S. Cl. CPC G16H 80/00; G16H 40/67; G16H 50/20

(57) ABSTRACT

A system for near-zero delay PEER to PEER communication between a Proctoring Unit (PU) and a Point of Care Unit (POCU) during remote proctoring PU clients and POCU clients in separate locations. The system comprises (a) a Main Host Server storing files and programs a STUN Server for generating and bridging connections between the PUs and the client POCUs; (b) client PU web browser control command actuators for controlling POCU client cameras, signal views and audio to be sent directly to the Video Server hosting the live video streaming software; (c) microphone, hdml camera and painter communication channels from PU to the client POCU via the Video Server; (d) an artificial intelligence unit configured for processing said live video and audio, sending processed data to said main host server (130). The artificial intelligence unit is configured for saving previously processed data and facilitating making medical manipulations according a predetermined protocol. The main host server (130) is configured for displaying said processed data at said Proctoring and Point of Care Units (110 and 120).

### CROSS-REFERENCE TO RELATED APPLICATIONS

The present application for patent is a Continuation-in-Part patent application of PCT Patent Application No. PCT/IL2022/050178 having International filing date of Feb. 15, 2022 and published as WO 2022/130395 on Jun. 23, 2022, which claims the benefit of priority of U.S. Provisional patent application 63/125,403 filed on Dec. 15, 2020, the contents of which are all incorporated herein by reference in their entirety.

### FIELD OF THE INVENTION

The present disclosure relates to a system for the remoting proctoring of medical procedures and training.

### BACKGROUND OF THE INVENTION

In light of the tremendous advancement and development occurring in many medical fields, such as structural heart interventions, over the last decade, and the usage of those techniques and devices to treat patients, surgeons, such as interventional cardiologists have had to learn and adopt complex new techniques that are not usually within the framework of their usual specialty training. As a result, the onus has fallen on the medical device companies that develop, test, produce and market these devices, to provide advanced training that is specific to their device. This support may include an umbrella of techniques, such as didactic learning, online training, working on simulators and animal studies etc. The final common pathway in almost all instances is through on-site proctoring support to the in-house operator/cardiologist. In some instances, the training is related only to the companies' specific device iteration but more often it involves teaching the team a whole new procedure as well as matters specifically concerning the device. Depending on the procedure/technique, support may be limited to only a few initial cases but for the more complex ones, it may involve the provision of clinical support in the long term on a regular basis. This is beneficial particularly where case volumes are not high and proctors gather very broad experience and share it with the local center as they generate their experience. As a result, the learning curve is rapid and complications can be avoided. There are multiple examples of this training technique, from TranCatheter Aortic Valve Replacement (TAVR) with multiple companies such as Edwards, Medtronic, Boston Scientific and Abbott, left atrial appendage (Abbott and Boston Scientific), Mitral valve repair (Abbott, Edwards, Neochord), Congenital defects (PFOs/ASDsNSDs/PDAs/Coarctation) and acquired other defects such as paravalvular leaks post MI VSDs (Abbott/Occlutech/Lifetech etc). This phenomenon has been present in other fields within cardiology, particularly electrophysiology where the implementation of advanced equipment (mapping, ablation, imaging etc.) has required external support for safe and effective utilization. The costs of this support are indirect and included in the purchase of the medical equipment passing the costs on to the hospitals and subsequently to the insurance or patient. In order to reduce the financial and logistic impact of this support, the support is shifted from physician proctors to clinical support technicians (Field clinical engineers in the US). The keys to the support provided, are that on-site proctor has access to the information needed for decision making and can shift his focus as needed. For most interventional cardiology this will include, fluoroscopy, echocardiography, hemodynamics, voice and visual input of the operators' movements and actions. This could all be replicated with imaging as above with high resolution associated with a voice input from the room, some cameras on the operators' hands and work space transferred to an off-site proctor all associated with the ability to provide verbal input to the operators. If all these data are supplied at adequate quality and resolution an off-site proctor could deliver an equivalent level of support as someone in the room. With the rapid expansion of these fields and devices, the ability for companies to physically place support in each site for every procedure has become increasingly challenging and will become impossible in the future. Off-site clinical support, while not replacing physical presence will remove a significant physical and financial load from the supporting companies. This may not replace first experiences, but for centers with basic knowledge and experience, off-site support will obviate the need for proctor traveling around the world and allow the clinical support team to be present at multiple sites on the same day from a single location. Furthermore, with the current covid-19 pandemic and the resulting travel restrictions and limitations, ongoing off-site clinical support could be provided, allowing medical centers to continue treating patients with the highest level of support and therefore care.

US application 20120191464A1 (InTouch Technologies Inc) discloses a proctoring system that includes a communication device coupled to a remote station. The remote station has a visual display that displays first information relating to an action that causes an effect on an object, and simultaneously displays second information relating to the effect on the object. The remote station includes at least one input device that allows a communication to be transmitted by an operator to the communication device. By way of example, during the deployment of a heart stent, a specialist doctor may remotely view real-time fluoroscopy imagery and patient hemodynamics. The specialist can remotely proctor medical personnel on the proper orientation and timing requirements for installing the stent.

U.S. Pat. No. 9,402,690 (Intuitive Surgical Operations Inc) discloses an apparatus is configured to show telestration in 3-D to a surgeon in real time. A proctor is shown one side of a stereo image pair, such that the proctor can draw a telestration line on the one side with an input device. Points of interest are identified for matching to the other side of the stereo image pair. In response to the identified points of interest, regions and features are identified and used to match the points of interest to the other side. Regions can be used to match the points of interest. Features of the first image can be matched to the second image and used to match the points of interest to the second image, for example when the confidence scores for the regions are below a threshold value. Constraints can be used to evaluate the matched points of interest, for example by excluding bad points.

In view of the prior art and given the various challenges described above, there is still an unmet long-felt need for a remote proctoring system run by human specialists, which enables control and communication by the proctor with various locations around the globe.

### SUMMARY OF THE INVENTION

A general design is provided of a system for near-zero delay PEER to PEER communication between Proctoring Unit (PU) clients (110) and the Point of Care Unit (POCU) (120) clients during remote proctoring

The System Comprises
a. a Main Host Server (130), storing files and programs, for live bi-directional communication between the client PUs and the client POCUs
b. a STUN Server (140) for generating and bridging connections between the PUs and the client POCUs
c. an RTC module (150) for casting video and audio communication from the STUN Server to the clients
d. a Video Server (160) configured for hosting live audio and video streaming software from said client POCU
e. client PU web browser control command actuators for controlling POCU client cameras, signal views and audio to be sent directly to the Video Server hosting the live video streaming software
f. microphone, hdml camera and painter communication channels from PU to the client POCU via the Video Server;
a. an artif an artificial intelligence unit configured for processing said live video and audio, sending processed data to said main host server (130); said artificial intelligence unit configured for saving previously processed data and facilitating making medical manipulations according a predetermined protocol; said main host server (130) is configured for displaying said processed data at said Proctoring and Point Of Care Units (110 and 120).
g. artificial intelligence unit configured for processing said live video and audio, sending processed data to said main host server (130); said artificial intelligence unit configured for saving previously processed data and facilitating making medical manipulations according a predetermined protocol; said main host server (130) is configured for displaying said processed data at said Proctoring and Point Of Care Units (110 and 120).

Embodiments of the aforementioned system further comprise

connection from POCU to Video Server for 2-cam, x-ray, emo, eco channels (121)

POCU microphone connection to PU via Video Server (122)

VISCA-over-IP for camera data from POCU to PU via Video Server (123)

Embodiments of the system of claim 1-2 comprises PU microphone communication to POCU streamed through the STUN server to the Video Server (111)

Embodiments of the aforementioned system comprises connection for camera and painter from PU to the POCU via the Video Server (112) wherein painter is a transparent canvas layer and is streamed over the STUN server to the POCU client over the video from the POCU client the streaming over occurring in the Audio Video Server.

Further embodiments of the aforementioned system comprises bi-directional communication between PU and Main Host Server (131), bi-directional communication between STUN Server and Main Host Server (141) and bi-directional communication from Video Server to Main Host Server (141)

In the present invention the term STUN stands for Session Traversal Utilities for NAT.

Session Traversal Utilities for NAT is a standard method of NAT traversal used in WebRTC.

The definition is in IETF RFC 5389.

A STUN server is a server that runs on the public network and replies to incoming requests. The responses sent out include the public IP address the request was sent to him from. This effectively answers the question “what is my IP address?”

An RTC module is used for real time communication when casting video and audio communication from the STUN Server to the clients

It is a further object of the present invention to disclose a proctoring system comprising:
a. a point-of-care unit POCU;
b. a proctor unit PU;
   wherein the POCU and the PU are functionally inter-connected.

It is another object of the present invention to disclose any of the systems above, wherein the POCU comprises at least one system for monitoring the actions of a medical professional.

It is another object of the present invention to disclose any of the systems above, wherein the monitoring system is selected from a group consisting of audio systems and visual systems.

It is another object of the present invention to disclose any of the systems above, wherein the audio system is a microphone.

It is another object of the present invention to disclose any of the systems above, wherein the visual system is a camera.

It is another object of the present invention to disclose any of the systems above, wherein the camera is characterized as manual, semi-automatic, automatic, remote controlled and robotic.

It is another object of the present invention to disclose any of the systems above, wherein the POCU comprises at least one system for monitoring the status of a patient.

It is another object of the present invention to disclose any of the systems above, wherein the system is selected from a group consisting of a medical monitor, a camera visual system and an audio system.

It is another object of the present invention to disclose any of the systems above, wherein the POCU comprises at least one system for presenting communication from the PU.

It is another object of the present invention to disclose any of the systems above, wherein the system for presenting communication selected from a group consisting of audio systems and visual systems.

It is another object of the present invention to disclose any of the systems above, wherein the system for presenting communication is selected from a group consisting of a display and a speaker.

It is another object of the present invention to disclose any of the systems above, wherein the the display system is an optical head-mounted display (OHMD).

It is another object of the present invention to disclose any of the systems above, wherein the display is helmet based or glass based.

It is another object of the present invention to disclose any of the systems above, wherein the display system is virtual reality, mixed reality, computer-mediated reality or augmented reality based.

It is another object of the present invention to disclose any of the systems above, wherein the display system is a 3-D glassware.

It is another object of the present invention to disclose any of the systems above, wherein the remote unit comprises at least one system for presenting monitored actions of a medical professional.

It is another object of the present invention to disclose any of the systems above, wherein the monitoring system is selected from a group consisting of audio systems and visual systems.

It is another object of the present invention to disclose any of the systems above, wherein the audio system is selected from a group consisting of a speaker and a headphone.

It is another object of the present invention to disclose any of the systems above, wherein the visual system is selected from a group consisting of a display and a projector.

It is another object of the present invention to disclose any of the systems above, wherein the remote unit comprises at least one system for presenting the status of the patient.

It is another object of the present invention to disclose any of the systems above, wherein the system is a medical monitor.

It is another object of the present invention to disclose any of the systems above, wherein the PU comprises at least one system for presenting communication from the POCU.

It is another object of the present invention to disclose any of the systems above, wherein the system for presenting communication is selected from a group consisting of a display and a speaker.

It is another object of the present invention to disclose any of the systems above, wherein the display system is an optical head-mounted display (OHMD).

It is another object of the present invention to disclose any of the systems above, wherein the display is selected from a group consisting of a helmet-based display and a glass-based display.

It is another object of the present invention to disclose any of the systems above, wherein the display system is a mixed reality, computer-mediated reality or augmented reality.

It is another object of the present invention to disclose any of the systems above, wherein the display system is a 3-D glassware.

It is another object of the present invention to disclose any of the systems above, wherein the proctor unit comprises a system for controlling at least one system situated in the POC unit.

It is another object of the present invention to disclose any of the systems above, wherein the remote-controlled unit is selected from a group consisting of a camera and a laser pointer.

It is another object of the present invention to disclose any of the systems above, wherein the control system is selected from a group consisting of a keyboard, a joystick, an eye tracker.

It is the object of the present invention to disclose a system characterized by:
a. a point-of-care unit POCU;
b. a proctor unit PU;
   wherein the POCU and the PU are characterized as being interconnected.

It is another object of the present invention to disclose any of the systems above, wherein the POCU is characterized as monitoring the actions of a medical professional.

It is another object of the present invention to disclose any of the systems above, wherein the monitoring system is selected from a group consisting of audio systems and visual systems.

It is another object of the present invention to disclose any of the systems above, wherein the audio system is a microphone.

It is another object of the present invention to disclose any of the systems above, wherein the visual system is a camera.

It is another object of the present invention to disclose any of the systems above, wherein the camera is characterized as manual, semi-automatic, automatic, remote controlled or robotic.

It is another object of the present invention to disclose any of the systems above, wherein the POCU is characterized as monitoring the status of the patient.

It is another object of the present invention to disclose any of the systems above, wherein the system is a medical monitor.

It is another object of the present invention to disclose any of the systems above, wherein the POCU is characterized as presenting communication from the PU.

It is another object of the present invention to disclose any of the systems above, wherein the system for presenting communication is selected from a group consisting of a display, a projector and a speaker.

It is another object of the present invention to disclose any of the systems above, wherein the PU is characterized as presenting monitored actions of a medical professional.

It is another object of the present invention to disclose any of the systems above, wherein the monitoring system is selected from a group consisting of audio systems and visual systems.

It is another object of the present invention to disclose any of the systems above, wherein the audio system is a speaker.

It is another object of the present invention to disclose any of the systems above, wherein the visual system is characterized as a display or a projector.

It is another object of the present invention to disclose any of the systems above, wherein the display system is characterized as an optical head-mounted display (OHMD).

It is another object of the present invention to disclose any of the systems above, wherein the display is characterized as helmet based or glass based.

It is another object of the present invention to disclose any of the systems above, wherein the display system is characterized as virtual reality, mixed reality, computer-mediated reality or augmented reality.

It is another object of the present invention to disclose any of the systems above, wherein the display system is characterized as a 3-D glassware.

It is another object of the present invention to disclose any of the systems above, wherein the PU is characterized as presenting the status of the patient.

It is another object of the present invention to disclose any of the systems above, wherein the system for presenting the status of the patient is characterized as a medical monitor.

It is another object of the present invention to disclose any of the systems above, wherein the PU is characterized as presenting communication from the POCU.

It is another object of the present invention to disclose any of the systems above, wherein the system for presenting communication is selected from a group consisting of a display and a speaker.

It is another object of the present invention to disclose any of the systems above, wherein the display system is characterized as an optical head-mounted display (OHMD).

It is another object of the present invention to disclose any of the systems above, wherein the display is characterized as helmet based or glass based.

It is another object of the present invention to disclose any of the systems above, wherein the display system is characterized as virtual reality, mixed reality, computer-mediated reality or augmented reality.

It is another object of the present invention to disclose any of the systems above, wherein the display system is characterized as a 3-D glassware.

It is another object of the present invention to disclose any of the systems above, wherein the PU is characterized as controlling at least one system situated in the POCU.

It is another object of the present invention to disclose any of the systems above, wherein the remote-controlled unit is characterized as a camera or a laser pointer.

It is another object of the present invention to disclose any of the systems above, wherein the control system is characterized as a keyboard, a joystick or an eye tracker.

It is the object of the present application to present a method of proctoring, comprising steps of
a. obtaining the proctoring system of claim 1 or claim 31;
b. connecting at least one point-of-care unit POCU and at least one proctor unit PU;
c. activating the units;
d. transferring data from the POCU to the PU;
e. displaying the data for at least one proctor;

It is another object of the present application is to present a method of proctoring as disclosed above, wherein the step of transferring data comprising transferring data characterized as selected from a group consisting of visual data, audio data and patient data.

It is another object of the present application is to present a method of proctoring as disclosed above, additionally comprising steps of Transferring information, instructions data from the PU to the POCU and displaying the data for at least one professional in the PU.

A method of remote proctoring with near zero delay comprising steps of
a. obtaining the proctoring system of claim 1
b. connecting at least one said point-of-care unit POCU and at least one said proctor unit PU;
c. activating said units;
d. transferring data from said POCU to said PU;
e. displaying said data for at least one proctor;
said data is selected from a group consisting of visual data, audio data and patient data transferring information and instructions from said PU to said POCU and displaying said data for at least one professional in said PU.

### BRIEF DESCRIPTION OF THE FIGURES

The accompanying drawings, which are included to provide a further understanding of the invention and are incorporated in and constitute a part of this specification, illustrate embodiments of the invention and together with the description serve to explain the principles of the invention.

FIG. 1—is a schematic presentation of a remote proctoring system;

FIG. 2—is a schematic presentation of further aspects and details of the present invention; and

FIG. 3 illustrates a remote proctoring system with an artificial intelligence unit.

### DETAILED DESCRIPTION OF THE PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a system for remote proctoring, supervising, controlling and communicating various operation rooms in different locations from one operational control room. A system for a near zero delay Peer to Peer communication during remote proctoring between proctoring clients and point of care clients is provided as described below.

The system of the present invention harnesses remote control systems by Adder (KVM and DATAVIDEO). Based on detecting signal transmission.

As used herein after, the term “remote proctoring” refers to the action of accompanying and guiding a medical procedure from location A, where the medical procedure occurs in location B. The remote proctoring process is carried out by the system of the present invention using telecommunication systems. The proctor could be an employee of a medical device company, guiding medical professionals, such as surgeons, on the correct use of a medical device or a senior medical professional directing the application of a new surgical technique during a a procedure. The proctor of the present invention is located in a central operational control room (referred to as “cockpit”), where he/she can optionally be connected by known telecommunication technologies to various medical centers (such as hospitals) around the world, and from where he/she can guide, assist or/or consult other health specialists during medical procedures.

In many cases, Proctoring has been defined as a ‘process through which skills and/or knowledge that a provider asserts he/she already possesses are confirmed’, to differentiate proctoring from other similar processes, such as training or treating.

The ‘clinical privileges’ of the proctor has defined by different relevant medical organization, such as a hospital (https://www.aagl.org/wp-content/uploads/2013/10/AAGL-privileging-guidelines-for-robotic-assisted-laparoscopy-for-member-comment.pdf, https://www.asge.org/docs/default-source/education/practice_guidelines/guidelines_privileging_credentialing_proctoring.pdf?sfvrsn=a1d5e851_8, https://www.aafp.org/about/policies/all/clinical-proctoring.html):
The proctor must have relevant clinical privileges in their relevant institution, but may not have to have the same in the institution where the surgery is taking place.
The proctor cannot be responsible for the treatment of the patient or for any part of the surgery (or be designated as an assistant surgeon).
In some cases, the proctor is required to be ‘independent’ to best observe and evaluate the applicant surgeon's action and overall performance, in a report submitted to the relevant person(s) in applicant's institution.

In addition, many hospitals and medical organizations, such as the American Society for Gastrointestinal Endoscopy, have specific and concrete guidelines for the selection and actions of a proctor.

As used herein after, the term “about” refers to any value being up to 25% lower or greater the defined measure.

The present invention provides a system for remote proctoring of medical procedures from one central operational control room (cockpit).

In one preferred embodiment of the present invention, the cockpit enables the proctor to supervise and communicate with several different operation rooms (OR) along the country (or in different countries). The proctor can control and manage the ‘detection’ systems situated in the ORs, in order to maintain optional bidirectional audio-visual communication lines with the medical specialist performing the procedure. The two locations could be in separate areas in the same compound (such as separate rooms) or could be separated countries (and therefore separated by long distances).

In another preferred embodiment of the present invention, the delay range of the remote proctoring system is less than two seconds.

In another preferred embodiment of the present invention, the bandwidth for connecting remote destinations is about 100 mega. In some embodiments, the connection is a P2P-2L line.

The system is based on signals sourced from the medical/surgical operation and operating theater providing the remote viewer with a ‘description’ of the procedure, enabling supervision, observation, study/learning of the medical team. The signals can be generated by the medical systems (such as patient monitors) or audio/video systems (such as cameras and microphones) positioned in strategic positions in the OR.

The system is based on advanced technologies for the transferring of data, such as audio and video over a data connection (such as an internet line, at least 100 mb/s) with a delay of up to 2 seconds (<2 seconds). In some embodiments, the delay is of 1 seconds or less (<1 second).

In some embodiments, the system comprises:
1. Point-of-care unit POCU: situated in or near point of the procedure, such as the operating room or point of care operated by or for the medical professional performing the procedure.
2. Remote unit—Proctor unit PU (cockpit): situated remote from the ‘point of care’ unit. Operated by or for the remote professional (a.k.a. proctor).
3. Communication system CS: connecting the POCU and PU the data systems. The communication system may be based on commonly used, commercially available and existing technologies and systems. The communication system may be based on the internet, intranet, the world wide web (WWW), cellular networks etc.

The objective technical problem of the present invention is to provide a system enabling the remote proctor to select and observe information, images, video, medical imaging, sound, data being generated during operations within the point of care unit, and to communicate and send messages and instructions to the staff in the point of care unit. All this must be done in real time with zero delay.

It is therefore core to the present invention to provide and disclose a multi-channel system which enables real time zero delay communication and transfer of all types of data information, and communications between the proctor unit and the point of care unit.

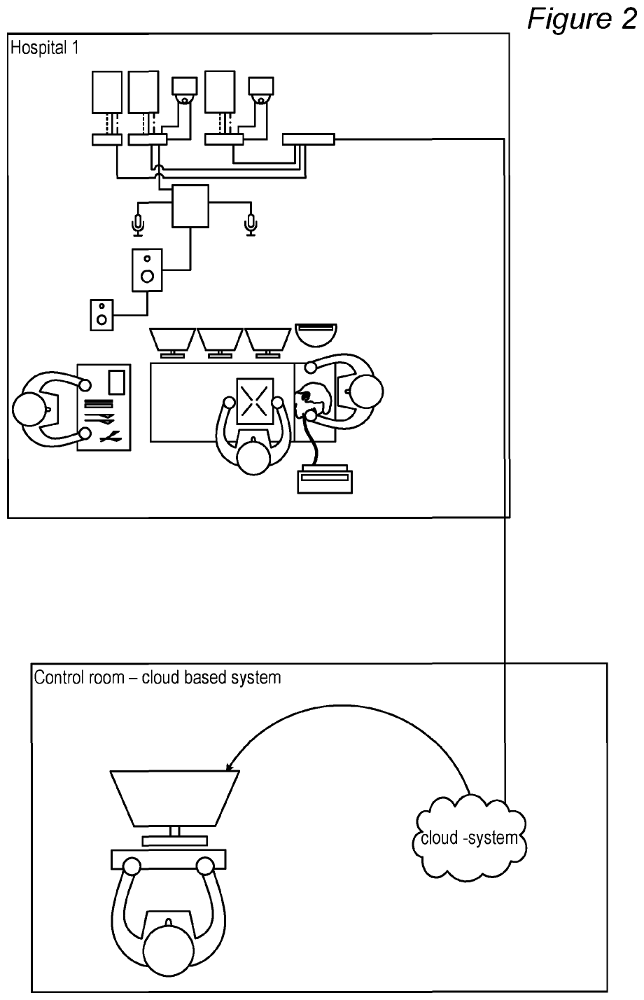

FIG. 2. shows a general design of the system for near-zero delay PEER to PEER communication between Proctoring Unit (PU) clients (110) and the Point Of Care Unit (POCU) (120) clients during remote proctoring is described below:

The Communication System (CS) comprises
h. a Main Host Server (130), storing files and programs, for live bi-directional communication between the client PUs and the client POCUs
i. a STUN Server (140) for generating and bridging connections between the PUs and the client POCUs
j. an RTC module (150) for casting video and audio communication from the STUN Server to the clients
k. a Video Server (160) configured for hosting live audio and video streaming software from said client POCU
l. client PU web browser control command actuators for controlling POCU client cameras, signal views and audio to be sent directly to the Video Server hosting the live video streaming software
m. microphone, hdml camera and painter communication channels from PU to the client POCU via the Video Server

Embodiments of the aforementioned system further comprises

connection from POCU to Video Server for 2-cam, x-ray, emo, eco channels (121)

POCU microphone connection to PU via Video Server (122)

VISCA-over-IP for camera data from POCU to PU via Video Server (123)

Embodiments of the system of claim 1-2 comprises PU microphone communication to POCU streamed through the STUN server to the Video Server (111)

Embodiments of the aforementioned system comprises connection for camera and painter from PU to the POCU via the Video Server (112) wherein painter is a transparent canvas layer and is streamed over the STUN server to the POCU client over the video from the POCU client the streaming over occurring in the Audio Video Server.

Further embodiments of the aforementioned system comprises bi-directional communication between PU and Main Host Server (131), bi-directional communication between STUN Server and Main Host Server (141) and bi-directional communication from Video Server to Main Host Server (141)

In the present invention the term STUN stands for Session Traversal Utilities for NAT.

Session Traversal Utilities for NAT is a standard method of NAT traversal used in WebRTC. The definition is in IETF RFC 5389.

A STUN server is a server that runs on the public network and replies to incoming requests. The responses sent out include the public IP address the request was sent from. This effectively answers the question “what is my IP address?”

An RTC module is used for real time communication when casting video and audio communication from the STUN Server to the clients

Point-of-Care Unit (POCU):

The POCU is a point-of-care system that enables the connection and interface of the medical professional or operator with the proctor (or supervisor). In some embodiments, the POCU enables the staff to interface (simultaneously or sequentially) with multiple proctors (or PUs). The POCU comprises modules for the detection of various inputs of the area, such as audio and video, and the status of the patient (such as the patients vital signs), with a control and display system, including cameras (inc. robotics), floro (reference+live), hemo-dynamics, Eco, IVUS, surgical glasses, 2- and 3-dimentional.

The camera could be static camera, a “robotic” (or automatic) camera (such as PTZ) or remote-controlled camera, enabling observation of the procedure. The camera(s) are strategically positioned to enable the proctor to best observe the procedure, such as positioning the camera over the operation and over the area used for the preparation of any additional tools or devises. The camera could be controlled using various technologies, such as a keyboard, joystick, voice recognition, gesture recognition, touchpad, eye tracker etc. The camera could be operated by a person near the patient (situated in the POCU) or by the proctor (situated in the PU), via the PU and CS.

The audio can be transmitted and/or captured by positioning an audio capture technology, such as a microphone, in the vicinity of the surgical perfectional(s), enabling continued communication with the proctor(s). The audio communication can be two-way by positioning a speaker (or headset) in the POCU.

In some embodiments, the POCU also comprises the means for connecting other systems used during the procedure, such as monitoring systems (such as medical monitors) and surgical systems (such as other system used during the procedure to assist the operator, such as lasers used during eye surgery, or the patients, such as a Membrane oxygenator or artificial lung).

In some embodiments, the POCU comprises technologies enabling the proctor to communicate with the perfectional conducting the procedure, such as presenting questions or instructions regarding the procedure. In some embodiments, the visual representation is general, such as a display screen, or personal display, such as an Optical head-mounted display (OHMD). The OHMD could be based on commercially available products or smart headsets, such as ‘Google glasses’, Microsoft HoloLens etc.

In some embodiments, the POCU comprises technologies presenting mixed reality, computer-mediated reality or augmented reality. In some embodiments, the POCU comprises means for immersive technologies enabling the presenting of the situation in the operating room by stimulating multiple senses, such as visual, audio, tactile (touch) etc. In some embodiments, the technologies are configured to detect the eye movement of the proctor, such as eye trackers.

In some embodiments, the POCU is enabled to display communications from the proctor, such as pictures (or marked pictures using an instrument, such as a stylus pen) or data. In some embodiments, the proctor can use a courser or indicator to mark a point of specific interest on a picture. In some embodiments, the POCU also comprises a pointer, such as a laser pointer, enabling the proctor to indicate or direct the operating team to a specific position of interest.

In some embodiments the POCU is configured for communicating with the PU unit by using the communication system via Computer-mediated communication (CMC) communicate, using various data or digital communications technologies or protocols, such as file format definitions and network communications protocol that uses TCP/IP to communicate between systems. Is some embodiments, the communication is characterized as videotelephony and comprises technologies for the reception and transmission of audio-video signals by users at different locations, for communication between people in real time. In some embodiments the communication conforms to various regulation and standards, such as Digital Imaging and Communications in Medicine (DICOM).

In some embodiments, the data system conforms to various regulations regarding security, data storage, authentication etc.

Proctor unit (PU):

The PU is a remote unit, connected to the ‘operation theater’, enabling the proctor to observe the procedure and communicate with the team conducting the medical procedure. The cockpit comprises a plurality of displays, such as monitors, presenting:
a visual representation of the procedure and of the actions of the professional conducting the procedure, such as captured by a plurality of cameras positioned in the operating room. In some embodiments, the visual representation is an Optical head-mounted display (OHMD), the OHMD could be based on commercially available smart headsets, such as ‘Google glasses’, Microsoft HoloLens etc.
An audio (or recording) of the sound of the operation theater, such as the sound of the procedure or the description made by the team conducting the procedure. The sounds are captured by a plurality of microphones positioned in the operating room.
Other data and signals generated by the procedure, often used as a means of monitoring the patient's status (such as the patient's vital perimeters of blood pressure, heart rate or pulse, body temperature and respiratory rate) can also be presented to the proctor. In some embodiments, medical results from previous states of treatment, such as during the diagnoses or screening of the patient, is also presented to the proctor, such as medical imaging (such as MRI or PET) or lab tests (such as biopsies or genetic testing).
Other data generated by other medical devices used in the procedure. This could be a systems used for conducting the operation (such as the high frequency thyroid ultrasound (HFUS) used to treat several thyroid gland conditions) or for assisting the patient during the procedure (such as the heart-lung machine or “the pump” used in coronary-bypass surgery).

The PU also comprises systems for directing the team conducting the medical procedure, such as microphones for communicating vocally and computer systems for generating graphical depictions (such as marking a specific point of interest on an image. In some embodiments the PU is enabled for the proctor to transmit relevant communication to the medical team, at the POCU. The communication could be audio or visual, such as a picture (such as a marked picture using an instrument such as a stylus) or data. The visual communication could be presented on the displays in the POCU.

In some embodiments the cockpit comprises a system for controlling or directing the detecting systems positioned near the patients, such as a keyboard, joystick, voice recognition, gesture recognition, touchpad eye tracker etc.

In some embodiments, the PU comprises a memory unit for recording the procedure (and all of the data generated during the procedure) in addition to the actions of the proctor in the PU.

In some embodiments, the PU comprises technologies presenting mixed reality, computer-mediated reality, virtual reality or augmented reality. In some embodiments, the cockpit comprised means for immersive technologies enabling the presenting of the situation in the operating room by stimulating multiple senses, such as visual, audio, tactile (touch) etc. In some embodiments, the technologies are configured to detect the eye movement of the proctor, such as eye trackers.

In an additional embodiment of the present invention, the PU is a central operational control room (“cockpit”) that is set in one specified location. This enables the proctor to arrive in a central position, to the cockpit, where he/she communicates with different sites around the globe.

In some embodiments the PU is configured for facilitating for communicating with the POCU by using the communication system via Computer-mediated communication (CMC). Is some embodiments, the PU is characterized as videotelephony and comprises technologies for the reception and transmission of audio-video signals by users at different locations, for communication between people in real time. In some embodiments the communication conforms to various regulation and standards, such as Digital Imaging and Communications in Medicine (DICOM).

In some embodiments, the data system conforms to various regulations regarding security, data storage, authentication etc.

Communication system (CS)

The CS is a system configured for facilitating the communication of the POCU and the PU (or multiple PUs) via Computer-mediated communication (CMC) based on a Web Socket protocol. The WebSocket protocol enables interaction between a web browser (or other client application) and a web server with lower overhead than half-duplex alternatives such as HTTP polling, facilitating real-time data transfer from and to the server. This is made possible by providing a standardized way for the server to send content to the client without being first requested by the client, and allowing messages to be passed back and forth while keeping the connection open. In this way, a two-way ongoing conversation can take place between the client and the server. Unlike HTTP. WebSocket provides full-duplex communication. Additionally, WebSocket enables streams of messages on top of TCP. TCP alone deals with streams of bytes with no inherent concept of a message. Before WebSocket, port 80 full-duplex communication was attainable using Comet channels; however, Comet implementation is nontrivial, and due to the TCP handshake and HTTP header overhead, it is inefficient for small messages. The Web Socket protocol aims to solve these problems without compromising the security assumptions of the web.

In some embodiments, the CMC is characterized as videotelephony and comprises technologies for the reception and transmission of audio-video signals by users at different locations, for communication between people in real time, such as the medical professionals (in the POC unit) and the proctor (in the remote unit). In some embodiments the communication conforms to various regulation and standards, such as Digital Imaging and Communications in Medicine (DICOM).

In some embodiments, the system comprises at least three sections:

Point-of-care unit POCU: situated in or near point of the procedure, such as the operating room or point of care operated by or for the medical professional performing the procedure.

Remote unit—Proctor unit PU (cockpit): situated remote from the ‘point of care’ unit. Operated by or for the remote professional (a.k.a. proctor).

Cloud-based unit CBU: Web Socket connecting the POCU and PU the data systems and containing processor, configured to provide computer processing to the PU and POCU. The CBU system may be based on commonly used, commercially available and existing technologies and systems. The CBU may be based on the internet, intranet, the world wide web (WWW), cellular networks etc.

Point-of-care unit (POCU):

The POCU is a point-of-care system that enables the connection and interface of the medical professional or operator with the proctor (or supervisor). In some embodiments, the POCU enables the staff to interface (simultaneously or sequentially) with multiple proctors (or PUs). The POCU comprises modules for the detection of various inputs of the area, such as audio and video, and the status of the patient (such as the patients vital signs), with a control and display system, including cameras (inc. robotics), floro (reference+live), hemo-dynamics, Eco, IVUS, surgical glasses, 2- and 3-dimentional.

The camera could be static camera, a “robotic” (or automatic) camera (such as PTZ) or remote-controlled camera, enabling observation of the procedure. The camera(s) are strategically positioned to enable the proctor to best observe the procedure, such as positioning the camera over the operation and over the area used for the preparation of any additional tools or devises. The camera could be controlled using various technologies, such as a keyboard, joystick, voice recognition, gesture recognition, touchpad, eye tracker etc. The camera could be operated by a person near the patient (situated in the POCU) or by the proctor (situated in the PU), via the PU and CBU.

The audio can be transmitted and/or captured by positioning an audio capture technology, such as a microphone, in the vicinity of the surgical perfectional(s), enabling continued communication with the proctor(s). The audio communication can be two-way by positioning a speaker (or headset) in the POCU.

In some embodiments, the POCU also comprises the means for connecting other systems used during the procedure, such as monitoring systems (such as medical monitors) and surgical systems (such as other system used during the procedure to assist the operator, such as lasers used during eye surgery, or the patient, such as a Membrane oxygenator or artificial lung).

In some embodiments, the POCU comprises technologies enabling the proctor to communicate with the professional conducting the procedure, such as presenting questions or instructions regarding the procedure. In some embodiments, the visual representation is general, such as a display screen, or personal display, such as an Optical head-mounted display (OHMD). The OHMD could be based on commercially available products or smart headsets, such as ‘Google glasses’, Microsoft HoloLens etc.

In some embodiments, the POCU comprises technologies presenting mixed reality, computer-mediated reality or augmented reality. In some embodiments, the POCU comprises means for immersive technologies enabling the presenting of the situation in the operating room by stimulating multiple senses, such as visual, audio, tactile (touch) etc. In some embodiments, the technologies are configured to detect the eye movement of the proctor, such as eye trackers.

In some embodiments, the POCU is enabled to display communications from the proctor, such as pictures (or marked pictures using an instrument such as a stylus) or data. In some embodiments, the proctor can use a courser or indicator to mark a point of specific interest on a picture. In some embodiments, the POCU also comprises a pointer, such as a laser pointer, enabling the proctor to indicate or direct the operating team to a specific position of interest.

In some embodiments the POCU is configured for communicating with the PU unit by using the communication system via Computer-mediated communication (CMC) communicate, using various data or digital communications technologies or protocols, such as file format definitions and network communications protocol that uses TCP/IP to communicate between systems. Is some embodiments, the communication is characterized as videotelephony and comprises technologies for the reception and transmission of audio-video signals by users at different locations, for communication between people in real time. In some embodiments the communication conforms to various regulation and standards, such as Digital Imaging and Communications in Medicine (DICOM).

In some embodiments, the data system conforms to various regulations regarding security, data storage, authentication etc.

Proctor unit (PU):

The PU is a remote unit, connected to the ‘operation theater’, enabling the proctor to observe the procedure and communicate with the team conducting the medical procedure. The cockpit comprises a plurality of displays, such as monitors, presenting:
a visual representation of the procedure and of the actions of the professional conducting the procedure, such as captured by a plurality of cameras positioned in the operating room. In some embodiments, the visual representation is an Optical head-mounted display (OHMD), the OHMD could be based on commercially available smart headsets, such as ‘Google glasses’, Microsoft HoloLens etc.
An audio (or recording) of the sound of the operation theater, such as the sound of the procedure or the description made by the team conducting the procedure. The sounds are captured by a plurality of microphones positioned in the operating room.
Other data and signals generated by the procedure, often used as a means of monitoring the patient's status (such as the patients' vital perimeters of blood pressure, heart rate or pulse, body temperature and respiratory rate) can also be presented to the proctor. In some embodiments, medical results from previous states of treatment, such as during the diagnoses or screening of the patient, is also presented to the proctor, such as medical imaging (such as MRI or PET) or lab tests (such as biopsies or genetic testing).
Other data generated by other medical devices used in the procedure. This could be a systems used for conducting the operation (such as the high frequency thyroid ultrasound (HFUS) used to treat several thyroid gland conditions) or for assisting the patient during the procedure (such as the heart-lung machine or “the pump” used in coronary-bypass surgery).

The PU also comprises systems for directing the team conducting the medical procedure, such as microphones for communicating vocally and computer systems for generating graphical depictions (such as marking a specific point of interest on an image. In some embodiments the PU is enabled for the proctor to transmit relevant communication to the medical team, at the POCU. The communication could be audio or visual, such as a picture (such as a marked picture using an instrument such as a stylus pen) or data. The visual communication could be presented on the displays in the POCU.

In some embodiments the cockpit comprises a system for controlling or directing the detecting systems positioned near the patient, such as a keyboard, joystick, voice recognition, gesture recognition, touchpad eye tracker etc.

In some embodiments, the PU comprises a memory unit for recording the procedure (and all of the data generated during the procedure) in addition to the actions of the proctor in the PU.

In some embodiments, the PU comprises technologies presenting mixed reality, computer-mediated reality, virtual reality or augmented reality. In some embodiments, the cockpit comprised means for immersive technologies enabling the presenting of the situation in the operating room by stimulating multiple senses, such as visual, audio, tactile (touch) etc. In some embodiments, the technologies are configured to detect the eye movement of the proctor, such as eye trackers.

In an additional embodiment of the present invention, the PU is a central operational control room (“cockpit”) that is set in one specified location. This enables the proctor to arrive in a central position, to the cockpit, where he/she communicates with different sites around the globe.

In some embodiments the PU is configured for facilitating for communicating with the POCU by using the communication system via Computer-mediated communication (CMC). Is some embodiments, the PU is characterized as videotelephony and comprises technologies for the reception and transmission of audio-video signals by users at different locations, for communication between people in real time. In some embodiments the communication conforms to various regulation and standards, such as Digital Imaging and Communications in Medicine (DICOM).

In some embodiments, the data system conforms to various regulations regarding security, data storage, authentication etc.

Cloud-Based Unit (CBU)

The CBU is a system configured for facilitating the communication of the POCU and the PU (or multiple PUs) via Computer-mediated communication (CMC). The CMC could be a communication protocol (such as Internet Protocol, IP) or a communication channel, comprising protocols for connecting the two units, such as routing protocols (or ad hoc routing protocol) or file transfer protocols. In some embodiments the CBU is positioned in a cloud or a server, configured to be accessed by the PU and the POCU. In some embodiments, the CBU comprises a security protocol, complying with regulation regarding patient privacy.

Is some embodiments, the CMC is characterized as videotelephony and comprises technologies for the reception and transmission of audio-video signals by users at different locations, for communication between people in real time, such as the medical professionals (in the POCU) and the proctor (in the remote unit). In some embodiments the communication conforms to various regulation and standards, such as Digital Imaging and Communications in Medicine (DICOM).

In some embodiments, the CBU further comprises additional functionality, such as a database of medical data, a processor, configured for image analysis, ability to storing images and data from medical procedures. In some embodiments, the CBU is configured to store the impute from the POCU and the PU during medical procedures.

In some embodiments, the CBU comprises a processor configured to run machine learning or artificial intelligence algorithms to analyses the data stored during medical procedures and data stores from previously conducted cases.

The FDA approves conducting certain medical procedures, such as valve replacement surgery, only if the proctor is physically present in the site where the procedure takes place, or if the medical specialists have met with the proctor several times.

Not every site has significant senior and experienced staff necessary to supervise complicated and cutting-edge procedures. This necessitates the use of stall situated at other medical facilities, often situated in other cities (or states).

Emerging pandemics, such as Covid-19, can make traveling difficult, in spite of available and rapid transportation, due to often changing health conditions and regulations. In addition, proctoring enables improved supervision and savings related to travel and housing of visiting proctors.

### Example 1

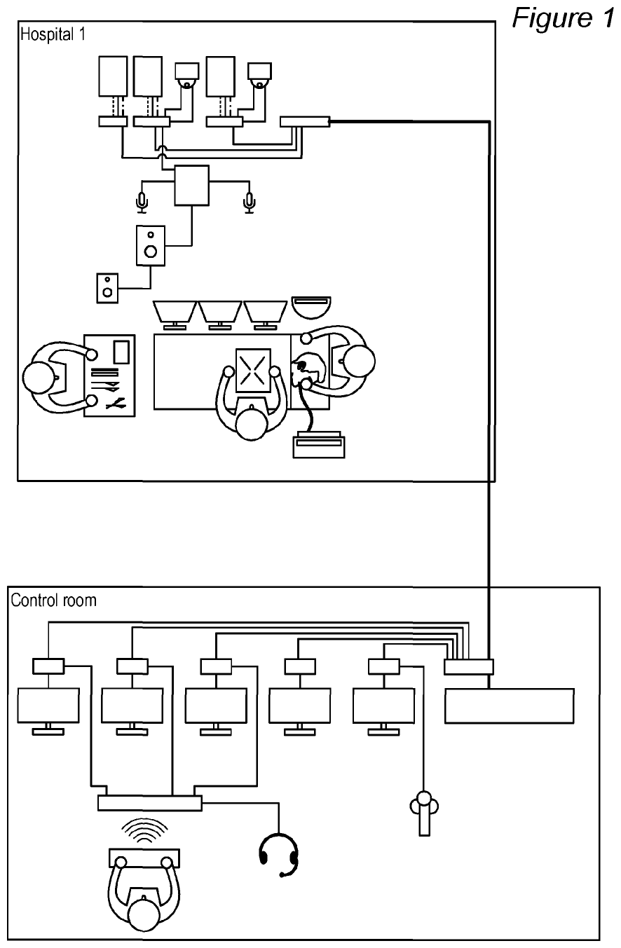

Reference in now made to FIG. 1 depicting an embodiment of the system, comprising:
POCU—Hospital 1, with Operating room (OP) room set up with:
   A plurality of cameras (Data video PTZ Camera 140) interconnected to a digital video/audio extender (Adder Allf 2020 TX) via DVI cable.
   A plurality of microphones (Microphone Deity S-MIC 25) and speakers, connected to an audio mixer (Yamaha MG10XU) via audio cable, connected to a digital video/audio extender (Adder Allf 2020 TX).
   The digital video/audio extender (Adder Allf 2020 TX) is connected to a network switch (Netgear Xsm 4324 SW) via ethernet.
   The network switch (Netgear Xsm 4324 SW) in connected to the other unit (such as a PU) via IP network.
PU—Control room:
   A plurality of displays, connected to a digital video/audio extender (Adder Allf 2020 RX), via Audio cable.
   A HSL Smart Multiview, connected to a digital video/audio extender (Adder Allf 2020 RX), via USB cable.
   A PTZ Camera Control Unit (DataVidoo RMC-180 CONTROLER) connected by RS232 to a digital video/audio extender (Adder Allf 2020 RX).
   An impute device (keyboard) and microphone-headset, connected via Bluetooth to the HSL Smart Multiview.

### Example 2

Eye surgery—3D microscope worn on the head of the surgeon is connected to a feed to the proctor in the remote unit. The proctor is able to view the procedure close up on a display, as seen by the surgeon. The proctor can relay instructions to the surgeon via a microphone in the remote unit and a speaker positioned in the POCU.

### Example 3

Cardiology (valve replacement surgery)—the surgeon is fitted with a head-set, comprising: (i) a 3-D camera that records the surgeon's point-of-view; (ii) a hand-free microphone, placed near the surgeon's mouth; and (iii) an earpiece, positioned on the surgeon's ear. The headset is connected to a wireless receiver positioned in the surgery, that transmits and receives over the internet. The proctor is fitted with a set of 3D-glasses (such as Google glasses) worn by the proctor, enabling the proctor to use these glasses instead of watching screens. The POCU additionally comprises a microphone and speaker, enabling the proctor to communicate with the surgeon.

### Example 4

according to one embodiment, the Cloud-based system comprises:
POCU—Hospital 1, with Operating room (OP) room set up with:
   A plurality of cameras (Data video PTZ Camera 140) interconnected to a digital video/audio extender (Adder Allf 2020 TX) via DVI cable.
   A plurality of microphones (Microphone Deity S-MIC 25) and speakers, connected to an audio mixer (Yamaha MG10XU) via audio cable, connected to a digital video/audio extender (Adder Allf 2020 TX).
   The digital video/audio extender (Adder Allf 2020 TX) is connected to a network switch (Netgear Xsm 4324 SW) via ethernet.
   The network switch (Netgear Xsm 4324 SW) in connected to the other unit (such as a PU) via IP network.
Cloud system or Cloud-based Unit (CBU), connecting the POCU and PU.
PU—Control room: A computer, configured to display the visual, audio and medical information from the POCU.

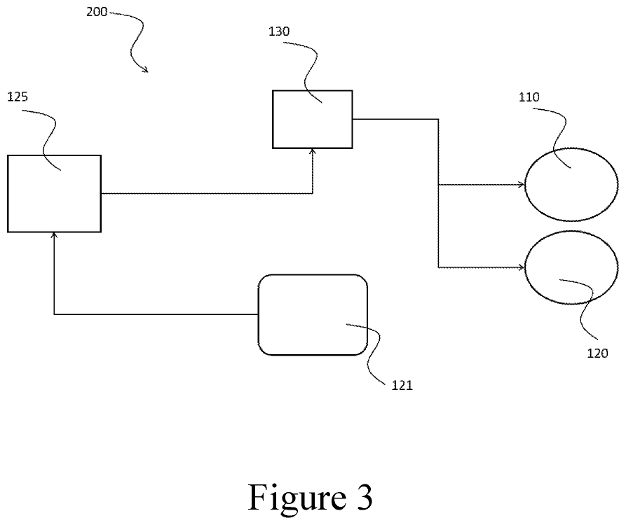

Reference is now made to FIG. 3 illustrating a system 200 including artificial intelligence (AI) unit 125. VMIX PC video signal 121 from point-of-care unit 110 is provided to AI unit 125. The aforesaid AI unit processes VMIX PC video signal 121 and sends obtained processed data to main host server 130. AI unit 125 stores the previously processed data and provides instruction relating to medical manipulation performed at point-of-care unit 110 in a real-time manner. The provided data are stored in main host server 130. Concurrently with it, provided instructions are displayed at point-of-care and proctoring units 110 and 120, respectively.

1. A system for near-zero delay PEER to PEER communication between Proctoring Unit (PU) clients (110) and the Point Of Care Unit (POCU) (120) clients during remote proctoring PU clients and POCU clients in separate locations, the system comprising
a. a Main Host Server (130) storing files and programs, for live bi-directional Web Socket communication between the PUs and the client POCUs
b. a STUN Server (140) for generating and bridging connections between the PUs and the client POCUs
c. an RTC module (150) for casting video and audio communication from the STUN Server to said clients
d. a Video Server (160) configured for hosting live audio and video streaming software from said client POCU
e. client PU web browser control command actuators for controlling POCU client cameras, signal views and audio to be sent directly to the Video Server hosting the live video streaming software
f. microphone, hdml camera and painter communication channels from PU to the client POCU via the Video Server;
g. an artificial intelligence unit configured for processing said live video and audio, sending processed data to said main host server (130); said artificial intelligence unit configured for saving previously processed data and facilitating making medical manipulations according a predetermined protocol;
said main host server (130) is configured for displaying said processed data at said Proctoring and Point Of Care Units (110 and 120).

2. The system of claim 1 comprising
a. connection from POCU to Video Server for 2-cam, x-ray, emo, eco channels (121)
b. POCU microphone connection to PU via Video Server (122)
c. VISCA-over-IP for camera data from POCU to PU via Video Server (123)

3. The system of claim 1 comprising PU microphone communication to POCU streamed through the STUN server to the Video Server (111)

4. The system of claim 1 comprising connection for camera and painter from PU to the POCU via the Video Server (112) wherein painter is a transparent canvas layer for streaming over the STUN server to the POCU client over the video from the POCU client, the stream over occurring in the Audio Video Server.

5. The system of claim 1 comprising bi-directional communication between PU and Main Host Server (131), bi-directional communication between STUN Server and Main Host Server (141) and bi-directional communication from Video Server to Main Host Server (141)

6. The system of claim 1, wherein said POCU has a plurality of audio systems and visual screen monitors and displays streaming communication through the STUN server to the Audio/Video Server.

7. The system of claim 3, wherein said audio system is a microphone and said visual system comprises at least one camera.

8. The system of claim 4, wherein said at least one camera is characterized as any of PTZ, manual, semi-automatic, automatic, remote controlled and robotic.

9. The system of claim 1, wherein said POCU comprises at least one system for medical monitoring the status of the patient.

10. The system of claim 9, wherein said medical monitoring system is selected from a group consisting of a medical monitor, a camera visual system and an audio system.

11. The system of claim 1, wherein said POCU comprises at least one system for presenting communication from said PU selected from a group consisting of audio systems and visual systems.

12. The system of claim 11, wherein said system for presenting communication comprises a display characterized by at least one of the following:
a. Screen display
b. a helmet-based
c. glasses-based unit
d. virtual reality, mixed reality, computer-mediated reality or augmented reality based platform.
e. a 3-D glasswear.

13. The system of claim 1, wherein said PU comprises at least one system for presenting communication from said POCU.

14. The system of claim 13, wherein said system for presenting communication is selected from a group consisting of a display and a speaker.

15. The system of claim 1, wherein said proctor unit comprises a system for controlling at least one system situated in said POCU.

16. The system of claim 1, wherein said client PU web browser control command actuators for controlling POCU client cameras is selected from a group consisting of a camera, laser pointer a keyboard, a joystick, an eye tracker, voice command actuator.

17. The system of claim 1, additionally comprising at least one cloud-based unit CBU.

18. The system of claim 17, wherein said CBU is configured to be accessed by said PU and said POCU and said CBU is configured for at least additional function, selected from a group consisting of image analysis, medical data storage, medical procedure storage.

19. A method of remote proctoring with near zero delay comprising steps of
a. obtaining the proctoring system of claim 1
b. connecting at least one said point-of-care unit POCU and at least one said proctor unit PU;
c. activating said units;
d. transferring data from said POCU to said PU;
e. displaying said data for at least one proctor;
said data is selected from a group consisting of visual data, audio data and patient data transferring information and instructions from said PU to said POCU and displaying said data for at least one professional in said PU.
